(19) United States
(12) Patent Application Publication
(10) Pub. No.: US 2009/0247647 A1
(43) Pub. Date: Oct. 1, 2009

(54) Method of Stimulating Proteoglycan Synthesis in Cells

(76) Inventors: Brian Genge, Columbia, SC (US); Licia Wu, Cayce, SC (US); Roy Wuthier, Columbia, SC (US)

Correspondence Address: NELSON MULLINS RILEY & SCARBOROUGH, LLP, 1320 MAIN STREET, 17TH FLOOR, COLUMBIA, SC, 29201, US

(21) Appl. No.: 12/224,055

(22) PCT Filed: Mar. 28, 2007
(86) PCT No.: PCT/US2007/007725

§ 371 Date: Dec. 9, 2008

Related U.S. Application Data
(60) Provisional application No. 60/786,782, filed on Mar. 28, 2006.

(51) Int. Cl. A61K 31/045; C12N 5/02; A61P 35/00; A61P 17/02

(52) U.S. Cl. 514/738; 435/375

(57) ABSTRACT

A method of stimulating the production of proteoglycans in a living animal cell comprising administering one or more Vitamin D₃ metabolites to the living cells is provided.

This application claims priority to U.S. Provisional Application Ser. No. 60/786,782, filed Mar. 28, 2006, entitled “Method for Preventing and Treating Osteoarthritis and Enhancing Wound Healing, Tissue Engineering, and Stem Cell Research.”

The present invention was developed with funding from the Office of Naval Research (Grant No. N00014-97-0806) and from the National Institute of Arthritis and Musculoskeletal and Skin Diseases (Grant Nos. AR18983 and AR42359). The U.S. Government has a paid-up license in this invention and the right in limited circumstances to require the patent owner to license others on reasonable terms as provided for by the terms of the above grants.

### BACKGROUND OF INVENTION

The present invention relates to the fields of osteoarthritis treatment and prevention, wound healing, cancer treatment and prevention, tissue engineering and stem cell research.

Skyrocketing health care costs and a growing population of overweight and/or aging subject population in the United States mandate that economical and improved treatments for diseases, particularly those that affect the elderly and obese (e.g., arthritis), are rapidly developed. Osteoarthritis, also known as degenerative arthritis or degenerative joint disease, is a condition in which low-grade inflammation results in pain in the joints, caused by wearing of the cartilage that covers and acts as a cushion inside the joints. As the bone surfaces become less well protected by cartilage, the subject experiences pain upon weight bearing activities, including walking and standing. Due to decreased movement because of the pain, regional muscles may atrophy, and ligaments may become more lax.

Osteoarthritis is a degenerative cartilage disease and is the most common form of arthritis and is the major cause of physical disability and mobility limitations in older people. Affecting nearly 21 million people in the United States, osteoarthritis accounts for 25% of visits to primary care physicians and half of all non-steroidal anti-inflammatory drug prescriptions. It is estimated that 80% of the population will have radiographic evidence of osteoarthritis by age 65, although only 60% of those will be symptomatic.

Existing treatments for osteoarthritis include treatment with non-steroidal anti-inflammatory drugs, local injections of glucocorticoid or hyaluronan, and in severe cases, join replacement surgery. There has been no cure for, or therapies effective against the onset of, osteoarthritis and these treatments have limited functional life, are largely palliative, and can be cost-prohibitive.

Subjects suffering from osteoarthritis often exhibit a Vitamin D₃ deficiency. Supplementation with Vitamin D₃ has previously been recognized as aiding in pain relief, but these treatments have not resulted in long-term improvement in the condition of the subject. Arebelovic, et al., Curr. Rheumatol Rep. 2005, 7(1):29-35

Considering the prevalence, morbidity and associated costs of osteoarthritis it is imperative that early stage interventions, preventive treatments and cures are developed. Further, it is essential to improving the current health care system and containing the associated costs of medical treatments that a paradigm shift takes place from the current treatment oriented society to a preventative oriented society.

Tissue engineering is a relatively new development in biomedical research in which an understanding of how living cells function will ultimately enable researchers to program and direct their cell's activity to promote the repair of damaged and diseased tissues. Coupling tissue engineering with the equally burgeoning and fledgling field of stem cell research, it will likely soon be possible to create living tissue equivalents, for example, liver, bone, and cartilage.

Tissue engineering has the potential to provide cost effective solutions for osteoarthritic diseases that could greatly increase the quality of life of the subject. For example, by using tissue engineering, isolated chondrocytes or mesenchymal stem cells could be grown in an anatomically shaped matrix. With the correct combination of type, amount and timing of addition of growth factors, an autologous living cartilage equivalent could be grown in the laboratory then implanted into the body to replace diseased articular cartilage, thus restoring normal joint function.

Articular cartilage is an essential component of the musculoskeletal system. Its primary functions include covering the contact surfaces of synovial joints to reduce friction during motion and absorbing compressive forces during load bearing. Articular cartilage within the joint is composed of specialized connective tissue that distributes load and decreases friction. Articular cartilage consists mainly of an extracellular matrix (made of type II collagen, proteoglycan), chondrocytes, and Water. More specifically, the cartilage matrix components by weight include water (60-80%), several types of collagen (10-20%), lipids, enzymes and proteoglycans (10-15%). The chondrocytes are only about 5% of the wet weight of cartilage but control the synthesis and maintenance of the matrix components.

Proteoglycans represent a special class of glycoproteins that are heavily glycosylated. They consist of a core protein with one or more covalently attached glycosaminoglycan chain(s). These glycosaminoglycan (GAG) chains are long, linear carbohydrate polymers that are negatively charged under physiological conditions, due to the occurrence of sulphate and uronic acid groups. Proteoglycans are a major component of the animal extracellular matrix, the ‘filler’ substance existing between cells in an organism. Here they form large complexes, both to other proteoglycans, to hyaluronic acid, and to fibrous matrix proteins (such as collagen). They are also involved in binding cations (such as sodium, potassium and calcium) and water, and also regulating the movement of molecules through the matrix. Evidence also shows they can affect the activity and stability of proteins and signalling molecules within the matrix. Individual functions of proteoglycans can be attributed to either the protein core or the attached GAG chain.

Negatively charged proteoglycans can bind to and cross-link positively charged collagen fibrils in the matrix. In combination with type II collagen, proteoglycans are responsible for the viscoelastic, compressive and tensile strength properties of articular cartilage. Because they are highly negatively charged, proteoglycans regulate matrix hydration by trapping and holding water and ions and thereby increasing the osmolarity of the extracellular matrix. This results in swelling pressure within the cartilage that imparts its incredible viscoelastic properties. Proteoglycans also provide an important diffusion barrier that buffers these cells from external influences.

The onset of osteoarthritis is thought to be associated primarily with biomolecular changes in the cartilage tissue. One major hypothesis is that the loss of proteoglycan from the extracellular matrix has is an initiating event in the onset of osteoarthritis. For example, studies by Maroudas et al., Biorheology 1985; 22:159-69, compared human cartilage from normal and osteoarthritic joints with respect to swelling pressure. Osteoarthritic cartilage was less able to resist water loss under a given applied pressure than normal cartilage and this decreased performance was attributed to a loss of part of the proteoglycans from the tissue. Borella, et al., Agents Actions, 1991; 34:220-2, later showed in mice that proteoglycan loss and subsequent replenishment in articular cartilage after a mild arthritic insult followed by overproduction of proteoglycan synthesis facilitated full cartilage repair in mice. More recently, in a feasibility study to quantitate proteoglycan in human knee cartilage using magnetic resonance imaging, Wheaton et al., Acad. Radiol. 2004; 11:21-8, showed the loss of proteoglycan was correlated with early stage osteoarthritis. In fact, when proteoglycans are removed from cartilage, there is a ten-fold decrease in the compressive modulus according to Thornton, et al., Biochem J. 1989; 260:277-82. Thus, sufficient proteoglycan levels are requisite for normal cartilage function while loss of proteoglycans from articular cartilage can be associated with degenerative changes of osteoarthritis.

Proteoglycan levels have also been implicated in the treatment of cancer and in the areas of wound healing. For example, it is known that the highly negative charge of proteoglycans can function as a diffusion barrier to cellular nutrients, ions, electrolytes, gases, proteins, growth factors and the like. It is also known that some forms of cancers and tumors require an inordinate supply of cellular nutrients in order to rapidly grow and divide. Anti-angiogenic factors, currently being used in some cancer therapies, can limit tumor growth by reducing the blood flow and supply of essential biomolecules into the tumor site. A natural barrier of proteoglycans may, therefore, effectively starve the tumor of essential nutrients to embalm the tumor in its own toxic metabolic by-products. If a diffusion barrier is created around the tumor by proteoglycans, depriving the cells of their requisite nutrients, then it may be possible to suffocate the rapidly growing tumor by enveloping it in a quasi-hermetic seal of proteogtycans.

Tissue engineering is a relatively new development in biomedical research in which an understanding of how living cells function will ultimately enable researchers to program and direct their activity to promote the repair of damaged and diseased tissues. Coupling tissue engineering with the equally burgeoning and fledgling field of stem cell research, it will likely soon be possible to create living tissue equivalents, for example, liver, bone, and cartilage.

It would be desirable, therefore, to develop a method of increasing proteoglycan levels to prevent and treat degenerative changes of osteoarthritis by increasing the synthesis of proteoglycans in cartilage, develop barriers to the growth of cancer cells, and improve wound treatment. A method of increasing proteoglycan levels may also be useful in advancing the fields of tissue engineering and stem cell research.

### SUMMARY OF INVENTION

In one aspect, the present invention is a method of stimulating the production of proteoglycan levels in living animal cells. The method includes administering one or more Vitamin D₃ metabolites to the living cells.

In another aspect, the invention is a method of preventing and/or treating osteoarthritis. The method includes administering one or more Vitamin D₃ metabolites to stimulate the production of proteoglycans in chondrocyte cells in a subject in need of treatment for osteoarthritis and having an affected joint area.

In yet another aspect, the invention is a method of treating cancer. The method includes administering one or more Vitamin D₃ metabolites to cancer cells to stimulate the production of proteoglyeans in the cancer cells.

In yet another aspect, the invention is a method of enhancing the rate of wound healing. The method includes administering one or more Vitamin D₃ metabolites to the wound.

In another aspect, the invention is the use of one or more Vitamin D₃ metabolites for the purpose of preparing a medicament for the prevention and treatment of osteoarthritis, cosmetic deficiencies and cancer, and for the enhancement of wound healing. In this aspect, the medicament includes one or more Vitamin D₃ metabolites and a pharmaceutically acceptable excipient.

These and other aspects of the invention will be understood and become apparent upon review of the specification by those having ordinary skill in the art.

### BRIEF DESCRIPTION OF THE DRAWINGS

FIG. 1 demonstrates stimulation of proteoglycan levels in growth plate chondrocyte cultures by 1,25(OH)₂ Vitamin D₃.

FIGS. 2A through 2D demonstrate time and dosage-dependent effects of 1,25(OH)₂D₃ and 24,25(OH)₂D₃ on proteoglycan levels of growth plate chondrocyte cultures compared in serum-containing DATP5 and serum-free HL-1 media.

FIG. 2A demonstrates the effect of 1,25(OH)₂D₃ with DATP5 medium.

FIG. 2B demonstrates the effect of 1,25(OH)₂D₃ with HL-1 medium.

FIG. 2C demonstrates the effect of 24,25(OH)₂D₃ with DATP5 medium.

FIG. 2D demonstrates effect of 24,25(OH)₂D₃ with HL-1 medium.

### DETAILED DESCRIPTION OF INVENTION

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

In one aspect, the invention is a method of stimulating the synthesis of proteoglycans in cells by administering one or more Vitamin D₃ metabolites to the cells.

As used herein, the term “Vitamin D₃ metabolites” refers to chemicals formed enzymatically from Vitamin D in humans. These compounds can influence a wide variety of reactions that regulate a large number of cellular functions. Those having ordinary skill in the art will recognize that the Vitamin D₃ metabolites may also be synthesized outside the body, and synthetic Vitamin D₃ metabolites are contemplated under the definition of Vitamin D₃ metabolites herein.

Similarly, as used herein, the term “Vitamin D₃ metabolites” shall include derivatives of Vitamin D₃ metabolites. Such derivatives are known to those having ordinary skill in the art.

Cholecalciferol is a form of Vitamin D, also called Vitamin D₃. 7-Dehydrocholesterol is the precursor of Vitamin D₃ and, in the body, only forms the vitamin after being exposed to UV radiation. After exposure to the sun, cholecalciferol is sent to the liver to be hydroxylated where it becomes 25-Hydroxyvitamin D₃ (a Vitamin D₃ metabolite). Next, it is sent to the kidney and once again hydroxylated becoming 1,25-Hydroxyvitamin D₃. 1,25-Hydroxyvitamin D₃ is the active hormone form of Vitamin D₃, for this reason Vitamin D is often referred to as a prohormone. It is structurally similar to steroids such as testosterone, cholesterol, and cortisol (though Vitamin D₃ itself is a secosteroid).

In the body, Vitamin D₃ is converted to 25-hydroxyvitamin D₃ in the liver by the hydroxylation of the 25 position. It is further hydroxylated to 1α,25-dihydroxyvitamin D₃ or 24R,25-dihydroxyvitamin D₃ by hydroxylation of the 1α- or 24-position in the kidney, respectively.

The most common Vitamin D₃ metabolites are 1,25 Hydroxyvitamin D₃, having the chemical structure:

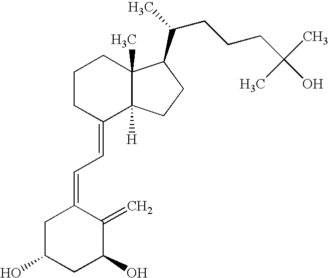

and 24,25 Hydroxyvitamin D₃, having the chemical structure:

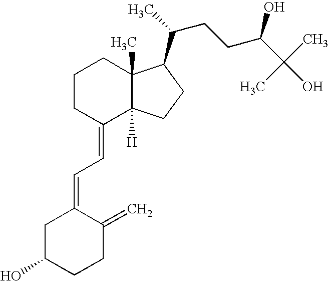

The one or more Vitamin D₃ metabolites may be administered to the cells to increase production of proteoglycans either in vitro, in vivo, or some combination thereof. In an exemplary embodiment, the Vitamin D₃ metabolites may be administered to a human subject as a liquid, a nutritional supplement, topically, and/or as a tablet or capsule.

Additionally, in some embodiments, the one or more Vitamin D₃ metabolites may be administered to one or more of normal, rapidly dividing, healthy, healing, and diseased cells. Exemplary cells contemplated for administration of the composition include one or more of chondrocytes (cartilage cells), synoviocytes (cells covering the internal surface of joints), pannocytes (cells found in cartilage erosions), and the like. Proteoglycans are associated with all cell types. Accordingly, the present composition and method may be administered to any cell in which increased production of proteoglycans may be useful.

For ease of reference, the present invention will be described with reference to administration to human subjects. It will be understood, however, that such descriptions are not limited to administration to humans, but will also include administration to other animals, such as mammals, unless explicitly stated otherwise.

The present method includes administering one or more Vitamin D₃ metabolites to the subject by administration means known in the art. Administration means contemplated as useful include one or more of topically, buccally, intranasally, orally, intravenously, intramuscularly, sublingually, and subcutaneously. Other administration means known in the art are also contemplated as useful in accordance with the present invention.

In some embodiments, it may be useful to include one or more Vitamin D₃ metabolites as a salt. Those having ordinary skill in the art will recognize the salts of the Vitamin D₃ metabolites.

The composition administered in accordance with the present invention may also include one or more of antihistamines, decongestants, expectorants, bronchodilators, beta-2-agonists, opoid agonists, opoid antagonists, antitussives, excipients, steroidal anti-inflammatory drug agents, non-steroidal anti-inflammatory drug agents, anticholinergic drug agents, beverages, beverage supplements, food supplements, and nutritional supplements.

In some embodiments, the composition may be an aqueous composition. The composition may also be nebulized or aerosolized.

The subject invention involves the use of a safe and effective amount of one or more Vitamin D₃ metabolites for the treatment of disorders in which an increase in the production of proteoglycans can treat the conditions. For example, the production of proteoglycans may be useful in the treatment of osteoarthritis, cancer, and wound healing, in humans and lower animals, especially humans. Additionally, the use of compositions including one or more Vitamin D₃ metabolites in tissue engineering and stem cell production is contemplated in accordance with the present invention.

An exemplary method of administering one or more Vitamin D₃ metabolites is topical, intranasal administration, e.g., with nose drops, nasal spray, or nasal mist inhalation. Other exemplary methods of administration include one or more of topical, bronchial administration by inhalation of vapor and/or mist or powder, orally, intravenously, intramuscularly, and subcutaneously.

In another embodiment, the one or more Vitamin D₃ metabolites may be administered in the form of a skin care product. For example, the one or more Vitamin D₃ metabolites may be incorporated into one or more of skin creams, butters, lotions, cleansers, solutions, lip creams and glosses, cosmetics, and natural extracts and oils known in the skin care industry to stimulate proteoglycan synthesis in the epidermal cells to help the skin retain moisture and to give a healthier appearance.

Added to lipstick formulations, the one or more Vitamin D₃ metabolites may stimulate proteoglycan synthesis in the epidermal cells of the lips to help the lips retain moisture and to give a fuller appearance.

In another embodiment, the one or more Vitamin D₃ metabolites may be administered in the form of nutritional supplements, supplements to foods, and supplements to beverages. For example, the Vitamin D₃ metabolites may be administered in one or more of energy bars, cereal products, genetically modified fruits, genetically modified grains, genetically modified vegetables, milk, juice, water, soft drinks, chewing gum, candies, cough drops, other beverages, and other foods.

Similarly, the one or more Vitamin D₃ metabolites may be added as a component in ionic liquids such as, for example, choline chloride:malonic acid. Those having ordinary skill in the art will recognize that ionic liquids are charged solvents that do not include water. Exemplary ionic liquids include those that may be beneficially metabolized by the body and are also often found naturally in the body. Additionally, exemplary ionic acids are those that are compatible with Vitamin D and safe for human consumption.

Other ingredients which may be incorporated in the present invention include safe and effective amounts of preservatives, e.g., benzalkonium chloride, thimerosal, phenylmercuric acetate; and acidulants, e.g., acetic acid, citric acid, lactic acid, and tartaric acid. The present invention may also include safe and effective amounts of isotonicity agents, e.g., salts, such as sodium chloride, and more preferably non-electrolyte isotonicity agents such as sorbitol, mannitol, and lower molecular weight polyethylene glycol.

Another aspect of the subject invention is a combination of a composition comprising an above-described composition including one or more of the Vitamin D₃ metabolites in a container comprising a means for topical administration of the present compositions to the eyes, nasal passages and sinuses, or bronchial passages and lungs. Preferred containers useful in such combinations include those comprising dropper means, spray means, or inhalation mist or powder means.

Containers comprising dropper means are useful for applying, as a liquid, either eye drops or nose drops topically to the eye or nasal passages, respectively. Such containers are well-known and commonly have such dropper means attached permanently or removably to the body of the container so that drops can be administered by inverting the container and/or by squeezing the container (the container being flexible). Another well-known dropper means is attached to a closure for the container and comprises a tube with a small hole in one end, the other end being open and attached to a flexible (e.g., rubber) bulb.

Containers comprising spray means are useful for applying a spray of liquid droplets topically directly to nasal passages. Well-known examples of such containers are flexible plastic containers having a spray nozzle fixedly attached thereto, the spray nozzle being designed for insertion into the nasal opening. When the container is squeezed, solution in the container is forced through the nozzle and emerges as a spray of droplets. Other well-known containers with spray means, e.g. pump sprays, nebulizers, or aerosol sprays, can also be used in a similar manner.

Containers comprising inhalation mist means are useful for applying a fine mist or powder topically to nasal passages and/or bronchial passages and lungs. Such inhalers provide a fine mist or powder which can be inhaled either through the nose or the mouth, depending on the design of the inhaler. Inhalers designed for providing a mist or powder to be inhaled through the nose are useful for topical administration of compositions to nasal passages and/or bronchial passages and lungs. Inhalers designed for providing a mist or powder to be inhaled through the mouth are useful for topical administration of compositions to bronchial passages and lungs. Various containers having inhalation mist or powder means as a part of or fixedly attached to the containers are well-known, e.g., squeeze containers, pump containers, and aerosols.

In the present method, a subject in need of prevention or treatment of a disorder in which an increase of proteoglycans would be effective in treating the disorder is treated with an amount of one or more Vitamin D₃ metabolites, where the amount of the one or more Vitamin D₃ metabolites provides a dosage or amount of the combination that is sufficient to constitute a treatment or prevention effective amount.

As used herein, an “effective amount” means the dose or amount to be administered to a subject and the frequency of administration to the subject which is readily determined by one or ordinary skill in the art, by the use of known techniques and by observing results obtained under analogous circumstances and has some therapeutic action. The dose or effective amount to be administered to a subject and the frequency of administration to the subject can be readily determined by one of ordinary skill in the art by the use of known techniques and by observing results obtained under analogous circumstances. In determining the effective amount or dose, a number of factors are considered by the attending diagnostician, including but not limited to, the potency and duration of action of the compounds used; the nature and severity of the illness to be treated as well as on the sex, age, weight, general health and individual responsiveness of the subject to be treated, and other relevant circumstances.

The phrase “therapeutically-effective” indicates the capability of an agent to prevent, or improve the severity of, the disorder, while avoiding adverse side effects typically associated with alternative therapies.

The one or more Vitamin D₃ metabolites can be supplied in the form of a novel therapeutic composition that is believed to be within the scope of the present invention.

When the one or more Vitamin D₃ metabolites are supplied along with a pharmaceutically acceptable carrier, a pharmaceutical composition is formed. A pharmaceutical composition of the present invention is directed to a composition suitable for the prevention or treatment of the disorders described herein. The pharmaceutical composition comprises at least a pharmaceutically acceptable carrier and one or more Vitamin D₃ metabolites. Pharmaceutically acceptable carriers include, but are not limited to, physiological saline, Ringer's, phosphate solution or buffer, buffered saline, and other carriers known in the art. Pharmaceutical compositions may also include stabilizers, anti-oxidants, colorants, and diluents. Pharmaceutically acceptable carriers and additives are chosen such that side effects from the pharmaceutical compound are minimized and the performance of the compound is not canceled or inhibited to such an extent that treatment is ineffective

The term “pharmacologically effective amount” shall mean that amount of a drug or pharmaceutical agent that will elicit the biological or medical response of a tissue, system, animal or human that is being sought by a researcher or clinician. This amount can be a therapeutically effective amount.

The term “pharmaceutically acceptable” is used herein to mean that the modified noun is appropriate for use in a pharmaceutical product. Pharmaceutically acceptable cations include metallic ions and organic ions. More preferred metallic ions include, but are not limited to, appropriate alkali metal salts, alkaline earth metal salts and other physiological acceptable metal ions. Exemplary ions include aluminum, calcium, lithium, magnesium, potassium, sodium and zinc in their usual valences. Preferred organic ions include protonated tertiary amines and quaternary ammonium cations, including in part, trimethylamine, diethylamine, N,N′-dibenzylethylenediamine, chloroprocaine, choline, diethanolamine, ethylenediamine, meglumine (N-methylglucamine) and procaine. Exemplary pharmaceutically acceptable acids include, without limitation, hydrochloric acid, hydroiodic acid, hydrobromic acid, phosphoric acid, sulfuric acid, methanesulfonic acid, acetic acid, formic acid, tartaric acid, maleic acid, malic acid, citric acid, isocitric acid, succinic acid, lactic acid, gluconic acid, glucuronic acid, pyruvic acid oxalacetic acid, fumaric acid, propionic acid, aspartic acid, glutamic acid, benzoic acid, and the like.

Also included in present invention are the isomeric forms and tautomers and the pharmaceutically-acceptable salts of Vitamin D₃ metabolites. Illustrative pharmaceutically acceptable salts are prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2 hydroxyethanesulfonic, sulfanilic, cyclohexylaminosulfonic, algenic, β-hydroxybutyric, galactaric and galacturonic acids.

Suitable pharmaceutically-acceptable base addition salts of compounds of the present invention include metallic ion salts and organic ion salts. More preferred metallic ion salts include, but are not limited to, appropriate alkali metal (Group IA) salts, alkaline earth metal (Group IIA) salts and other physiological acceptable metal ions. Such salts can be made from the ions of aluminum, calcium, lithium, magnesium, potassium, sodium and zinc. Preferred organic salts can be made from tertiary amines and quaternary ammonium salts, including in part, trimethylamine, diethylamine, N,N′-dibenzylethylenediamine, chloroprocaine, choline, diethanolamine, ethylenediamine, meglumine (N-methylglucamine) and procaine. All of the above salts can be prepared by those skilled in the art by conventional means from the corresponding compound of the present invention.

The terms “treating” or “to treat” means to alleviate symptoms, eliminate the causation either on a temporary or permanent basis, or to prevent or slow the appearance of symptoms. The term “treatment” includes alleviation, elimination of causation of or prevention of any of the diseases or disorders described above. Besides being useful for human treatment, these combinations are also useful for treatment of mammals, including horses, dogs, cats, rats, mice, sheep, pigs, etc.

The term “subject” for purposes of this application includes any animal. The animal is typically a human. A preferred subject is one in need of treatment or prevention of the disorders discussed herein.

For methods of prevention, the subject is any human or animal subject, and preferably is a subject that is in need of prevention and/or treatment of osteoarthritis, cancer, or any other disorder that may be effectively treated by increasing proteoglycan levels in the cells. The subject may be a human subject who is at risk of disorders such as those described above. The subject may be at risk due to genetic predisposition, sedentary lifestyle, diet, exposure to disorder-causing agents, exposure to pathogenic agents and the like.

The present pharmaceutical compositions may be administered enterally and/or parenterally. Parenteral administration includes subcutaneous, intramuscular, intradermal, intramammary, intravenous, and other administrative methods known in the art. Enteral administration includes solution, tablets, sustained release capsules, enteric coated capsules, syrups, beverages, foods, and other nutritional supplements. When administered, the present pharmaceutical composition may be at or near body temperature.

The phrase “therapeutically-effective” and “effective for the treatment, prevention, or inhibition,” are intended to qualify the amount of each agent for use in the therapy which will achieve the goal of increased proteoglycan levels, while avoiding adverse side effects typically associated with alternative therapies.

In particular, the Vitamin D₃ metabolites compositions of the present invention can be administered orally, for example, as tablets, coated tablets, dragees, troches, lozenges, aqueous or oily suspensions, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, maize starch, or alginic acid; binding agents, for example starch, gelatin or acacia, and lubricating agents, for example magnesium stearate, stearic acid or talc. The tablets may be uncoated or they may be coated by known techniques to delay disintegration and adsorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin, any of a variety of herbal extracts, milk, or olive oil.

Aqueous suspensions can be produced that contain the active materials in admixture with excipients suitable for the manufacture of aqueous suspensions. Such excipients are suspending agents, for example, sodium carboxymethylcellulose, methylcellulose, hydroxypropylmethyl-cellulose, sodium alginate, polyvinylpyrrolidone gum tragacanth and gum acacia; dispersing or wetting agents may be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate.

The aqueous suspensions may also contain one or more preservatives, for example, ethyl or n-propyl p-hydroxybenzoate, one or more coloring agents, one or more flavoring agents, or one or more sweetening agents, such as sucrose or saccharin.

Oily suspensions may be formulated by suspending the active ingredients in an omega-3 fatty acid, a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents, such as those set forth above, and flavoring agents may be added to provide a palatable oral preparation. These compositions may be preserved by the addition of an antioxidant such as ascorbic acid.

Dispersible powders and granules suitable for preparation of an aqueous suspension by the addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavoring and coloring agents, may also be present.

Syrups and elixirs containing the novel combination may be formulated with sweetening agents, for example glycerol, sorbitol or sucrose. Such formulations may also contain a demulcent, a preservative, and flavoring and coloring agents.

The subject compositions can also be administered parenterally, either subcutaneously, or intravenously, or intramuscularly, or intrasternally, or by infusion techniques, in the form of sterile injectable aqueous or olagenous suspensions. Such suspensions may be formulated according to the known art using those suitable dispersing or wetting agents and suspending agents which have been mentioned above, or other acceptable agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, n-3 polyunsaturated fatty acids may find use in the preparation of injectables;

The subject compositions can also be administered by inhalation, in the form of aerosols or solutions for nebulizers, or rectally, in the form of suppositories prepared by mixing the drug with a suitable non-irritating excipient which is solid at ordinary temperature but liquid at the rectal temperature and will therefore melt in the rectum to release the drug. Such materials are cocoa butter and poly-ethylene glycols.

The novel compositions can also be administered topically, in the form of creams, ointments, jellies, collyriums, solutions, patches, or suspensions.

Daily dosages can vary within wide limits and will be adjusted to the individual requirements in each particular case. In general, for administration to adults, an appropriate daily dosage has been described above, although the limits that were identified as being preferred may be exceeded if expedient. The daily dosage can be administered as a single dosage or in divided dosages.

Various delivery systems in addition to nutritional supplements include sprays, capsules, tablets, drops, and gelatin capsules, for example.

Those skilled in the art will appreciate that dosages may also be determined with guidance from Goodman & Goldman's The Pharmacological Basis of Therapeutics, Ninth Edition (1996), Appendix II, pp. 1707-1711.

In the present method, the amount of Vitamin D₃ metabolites that are used in the novel method of treatment preferably ranges from about 0.1 to about 2400 IU (expressed in terms of Vitamin D) (International Units) per day, more preferably between about 100 and about 1500 IU per day, and most preferably between about 150 and 1000 IU per day. As understood by those having ordinary skill in the art, an International Unit is a unit of measurement for the amount of a substance, based on measured biological activity (or effect). It is used for vitamins, hormones, some drugs, vaccines, blood products and similar biologically active substances. The precise definition of one IU differs from substance to substance and is established by international agreement. One IU of Vitamin D₃ (and its metabolites and derivatives) is the biological equivalent of 0.025 μg Vitamin D₃ (1140 μg exactly).

The amount of the one or more Vitamin D₃ metabolites that is used in the subject method may be an amount that is sufficient to cause an increase in the concentration of proteoglycans in cells that would benefit from an increase in the concentration of proteoglycans.

Those having ordinary skill in the art will recognize from the examples included herein that if the dose of one or more Vitamin D₃ metabolites surpasses beneficial levels, it will actually result in a decrease of proteoglycan concentration in the cells. In some applications, it may be desirable to limit the proteoglycan concentrations. For example, some cancer cells require expressions of proteoglycans in their surrounding matrix in order to spread. In these situations, specific inhibition of proteoglycan synthesis may serve to treat the disease. This inhibition may be induced by the present administration of one or more Vitamin D₃ metabolites.

The present method relates to the effective treatment and prevention of osteoarthritis, among other applications. In one embodiment, it is possible to reverse some of the degenerative changes of osteoarthritis by increasing the synthesis of proteoglycans by chondrocytes in cartilage. To this end, the present method involves administering nanomolar levels of 1,25(OH)₂ Vitamin D₃ (or 24,25(OH)₂ Vitamin D₃) to growth plate chondrocytes to increase proteoglycan content of the cultures. The treatment of osteoarthritis using one or more of Vitamin D₃ metabolites, Vitamin D₃ derivatives, and the synthetic analogues of Vitamin D₃ and Vitamin D₃ derivatives, can be done conveniently as a supplement to milk, soft drinks, sports drinks, bottled water, and the like, or as a tablet.

Vitamin D₃ metabolites are small molecules with oral bioavailability that, when included in the diet, may offer convenient, practical, economical and salubrious solutions to help prevent the onset of some cases of osteoarthritis. Alternatively, the Vitamin D₃ metabolites could be attached to a larger biopolymer, such as hyaluronic acid, chitosan, alginate, collagen, polyvinyl alcohols, polyacrylates, derivatives thereof, and combinations thereof, that is used to increase lubricity of the joint. While bound to the larger polymer, either covalently, ionically, hydrophobically, etc., the small molecule may be inert, but stored localized in the joint space for later use. As the larger polymer is degraded over time, it would release the small molecule, thus allowing it to become biologically active, and stimulating the surrounding cells to produce more proteoglycans, helping improve or maintain normal joint lubricity and function.

In one embodiment, the one or more Vitamin D₃ metabolites may be crosslinked to hyaluronic acid (or another biomaterial) and then injected directly to the afflicted joint space so the surrounding cells in need of treatment (for example, chondrocytes demonstrating evidence of osteoarthritis) would be exposed to the Vitamin D₃ metabolites. As the hyaluronic acid breaks down, the one or more Vitamin D₃ metabolites would be released locally, helping the natural production of proteoglycans in the cartilage.

In another embodiment of this invention, because proteoglycans are synthesized by a variety of cells following insult or injury to tissue, 1,25(OH)₂ Vitamin D₃ or 24,25(OH)₂ Vitamin D₃ may also have broad applications in the area of wound healing. For example, PRK, LASIK and LASEK eye surgery are becoming very popular treatments for vision correction. However, irregular healing after the operation as a result of scarring or poor matrix deposition can lead to hazing or other refractive errors. Novel approaches are being sought by the opthalmology community to modify the complex corneal wound healing cascade to suppress the subepithelial haze and regression that can take place after laser ablation. Current therapies such as administration of corticosteroids are non-specific and have potentially dangerous side effects. In one embodiment of the present invention, eye drops typically given to subjects following these laser vision correction procedures could include one or more Vitamin D₃ metabolites to expedite healing and mitigate and obviate some of these common adverse side effects of these procedures by increasing the proteoglycan expression during corneal wound healing.

Furthermore, Vitamin D₃, to stimulate proteoglycan production, in combination with retinoic acid (Vitamin A) or other growth factors to stimulate collagen production may be components of a liquid formulation that could be added to the eye drop-wise to improve vision or even give rise to “super-vision” without the use of surgery.

Moreover, the present one or more Vitamin D₃ metabolites may be applied to other wound sites to improve healing. Addition of the one or more Vitamin D₃ metabolites to the wound locations may increase proteoglycan production within the wounds, improving the rate of healing and reducing resultant scars.

It is also believed the current invention has application in cancer treatment. In one embodiment, the present method includes effectively starving the tumor of essential nutrients or embalming the tumor in its own toxic metabolic by-products by entombing it in a natural barrier of proteoglycans. If a diffusion barrier is created around the tumor by proteoglycans, depriving the cells of their requisite nutrients, then it may be possible to ‘suffocate’ or ‘starve’ the rapidly growing tumor by enveloping it in a ‘hermetic’ seal of proteoglycans. More specifically, the Vitamin D₃ metabolite, or its derivative described herein could be administered orally to the subject, or directly injected into or near the tumor site. This then could cause the tumor cells to secrete a matrix of proteoglycans that would then serve to envelop and limit the influx of essential nutrients and also the efflux of toxic cancer cellular metabolites, potentially killing the cancer cells or tumors such as adenomas, carcinomas, myelomas, gliomas, among others.

As shown herein, the Vitamin D₃ metabolites are effective in developing cells, and the effects are cell-stage specific. Accordingly, the Vitamin D₃ metabolites demonstrate functionality in programming and directing the differentiation of a variety of cell types, particularly stem cells. The present invention, therefore, will find use and application in controlling stem cell development, tissue engineering, and biomaterials research, among other applications.

Serum levels of 1,25(OH)₂D₃ are inversely correlated with Ca²⁺ levels. The present invention demonstrates that levels of 1,25(OH)₂D₃ cause increased proteoglycan levels in defined media. Thus, in a hypocalcemic state, proteoglycan would continue in anticipation of restoration of Ca²⁺ that would enable mineralization to resume. The specific increase in proteoglycan levels would increase water content and turgidity (the mechanical strength, because water is noncompressible) to the tissue in the absence of mineral deposition. This would explain expansion of proteoglycan levels in the absence of calcification during development of rickets.

The following experiments are provided to illustrate the present invention and are not intended to limit the scope of the invention. Furthermore, these experiments are not limited to the effect on chondrocytes but also relate to Vitamin D₃ effects in all cell types in all states of differentiation and apply to normal, rapidly dividing, healthy, healing or diseased cells and tissues that can be stimulated to produce proteoglycans by the Vitamin D₃ metabolites.

The following examples describe preferred embodiments of the invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples.

### EXAMPLES

### Example 1

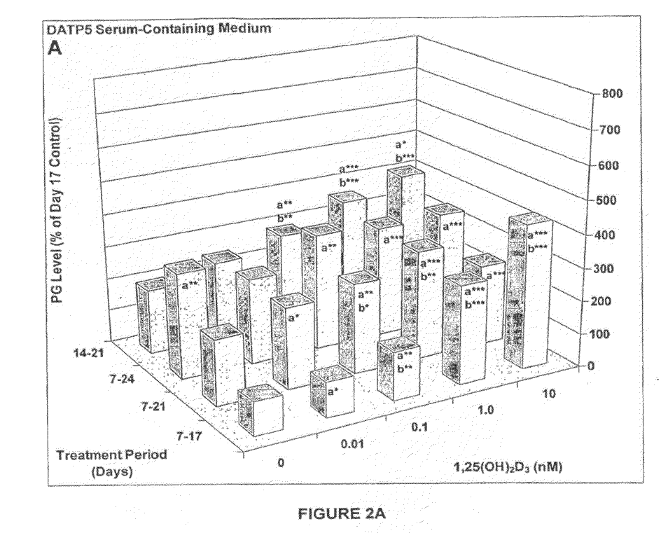

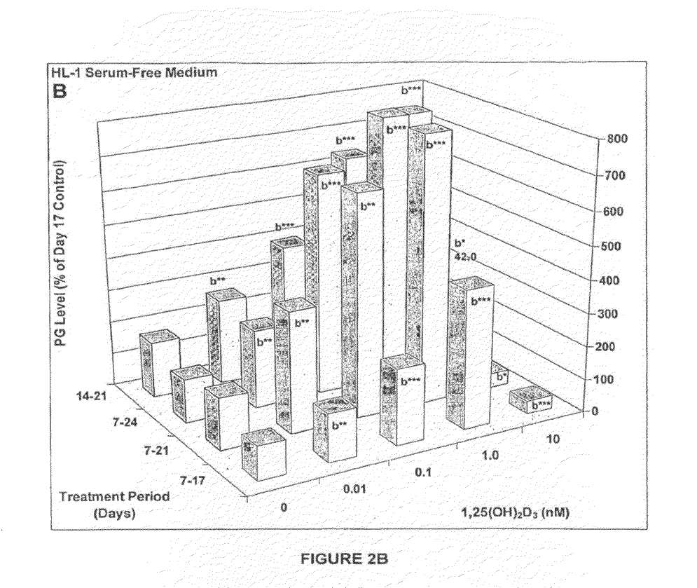

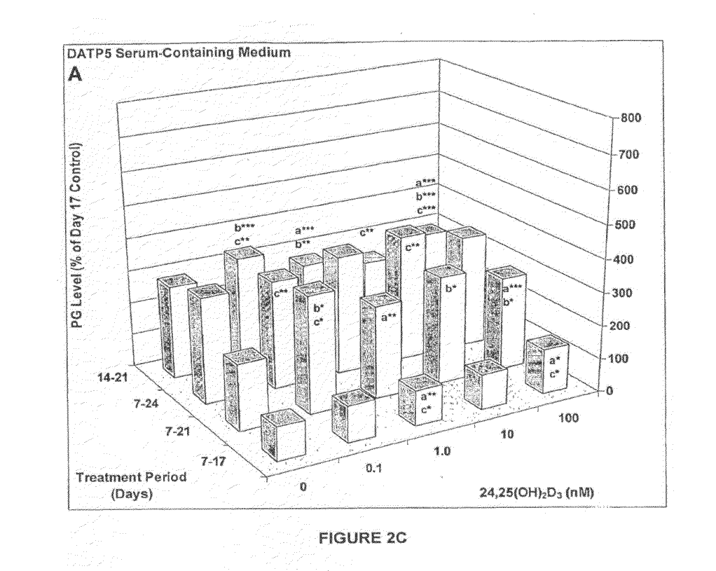

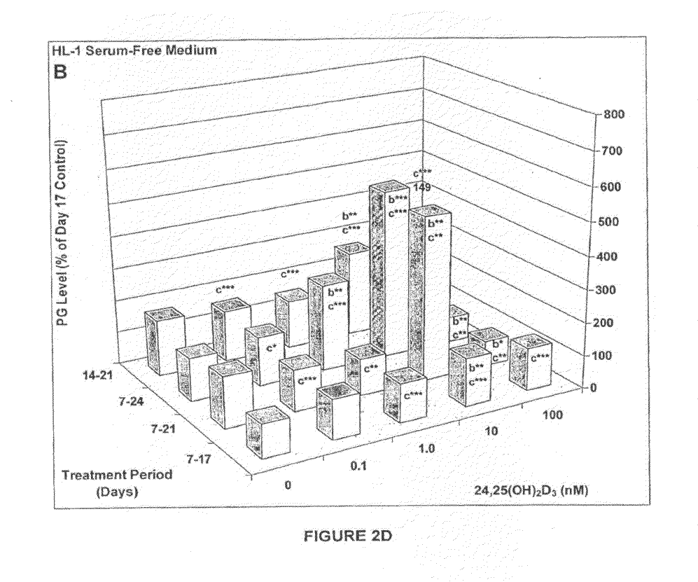

Time and dosage-dependent effects of 1,25(OH)₂ Vitamin D₃ and 24,25(OH)₂ Vitamin D₃ on primary cultures of pre- and post-confluent avian growth plate (GP) chondrocytes were examined. Cultures were grown either in a serum-containing culture medium designed to closely mimic normal GP extracellular fluid (DATP5) or a serum-free culture medium. A variety of cellular parameters were measured including proteoglycan (PG), lactate dehydrogenase (LDH) and alkaline phosphatase (ALP) activity and calcium and phosphate mineral deposition in the extracellular matrix were also measured. Results of the proteoglycan assays are shown in FIG. 2.

To develop the cell cultures, epiphyseal growth plates were dissected from the tibiae of a large number of 6-8-week-old hybrid broiler-strain chickens. The plates were pooled and the chondrocytes isolated. For these studies, the isolated cells were distributed into 320 identical primary cultures grown in 35 mm dishes in DMEM (2 ml per dish) with 10% fetal bovine serum for the first 3-4 days. Culture media were changed and a supplement of fresh ascorbic acid (25-50 μg/ml) added every 3-4 days for the duration of the experiments. From day 7 onward for 160 of the cultures, the medium was changed to DATP5 mineralization medium. To the other 160 dishes on day 7 the medium was changed to DMEM:HL-1 (1:1) and from day 10 onward to HL-1 serum-free medium (Biowhittaker, Walkersville, Md.). HL-1 chemically defined proprietary media contains transferrin, testosterone, sodium selenite, ethanolamine, saturated and unsaturated fatty acids, and stabilizing proteins (less than 30 μg/ml) and is supplemented with 10 mM β-glycerophosphate. DATP5 medium was prepared from DMEM basal medium by addition of eight amino acids, insulin (5 μg/ml)-transferrin (5 μg/ml)-selenite (5 ng/ml), and 5% defined FBS; it had a total inorganic phosphate (Pi) level of 1.9 mM (0.9 mM from OMEM+1 mM added Na₂HPO₄) and Ca²⁺ of 1.8 mM. All culture media contained 100 IU/ml of penicillin, 100 μg/ml streptomycin, and 0.25 μg/ml amphotericin B.

Administration of the vitamin D₃ metabolites was initiated either before the cells attained confluence (Day 7, pre-confluent tests), or after they had become confluent (Day 14, post-confluent tests), and continued as indicated. Stock solutions (2 mM) of the vitamin D₃ metabolites were prepared and further diluted in ethanol so that 2 μl of the test solution was added to 2 ml of culture medium.

To analyze biochemical activities, cells were harvested from the mm dishes in 1 ml TMT buffer (50 mM Tris, pH 7.5, 0.5 mM MgCl₂, 0.05% Triton X-100), the samples being sonicated in a water-bath. Aliquots were taken from each sample for analysis of the following seven parameters: alkaline phosphatase (ALP) and lactate dehydrogenase (LDH) activities, Hoechst DNA, Lowry protein, proteoglycan (PG) and Ca²⁺ and Pi mineral content. In brief, ALP activity was assayed at 37° C. in 750 mM AMP buffer, pH 10.3, containing 1 mM p-NPP and 0.25 mM MgCl₂ by monitoring the formation of p-nitrophenol for 5-10 minutes. ALP units were expressed as nanomoles of p-NPP hydrolyzed per minute, based on the extinction coefficient of p-nitrophenol of 18,450 M⁻¹cm⁻¹. Cellular and matrix protein content was analyzed using the method of O. H. Lowry, et al., J Biol Chem 1951; 193:265-275. Bovine serum albumin was used as a standard. Proteoglycan content of the cell matrix was analyzed using the dimethylmethylene blue reagent 11 measuring the absorbency difference at 520 and 589 nm. The assay is based on the ability of the sulfated glycosaminoglycans to bind to the cationic dye, dimethylmethylene blue, in solution. DNA content of TMT sonicates was assayed fluorometrically in Millipore F plates after cell lysis by freezing in distilled water then mixing with Hoechst 33258. Hoechst 33258 is a bisbenzimide DNA intercalator that excites in the near UV (350 nm) and emits in the blue region (450 nm). Hoechst 33258 binds to the AT rich regions of double stranded DNA and exhibits enhanced fluorescence under high ionic strength conditions.

This method allows for determining DNA and is not affected by contaminating protein or RNA. For determination of LDH activity and mineral (calcium and phosphate) content, the TMT sonicates were centrifuged for 1 h to separate insoluble proteins and minerals. LDH activity in the TMT supernatant was assayed at 37° C. in 0.2 M Tris buffer, pH 7.5, containing 0.2% Triton X-100, 0.15 mM NADH, and 1 mM sodium pyruvate. To extract mineral ions, 0.1 N HCl was first added to the culture dish, and then a precalculated aliquot was transferred to the sediment in the centrifuge tube equivalent to 1 ml/dish. After vortexing and standing at room temperature for 1 hour, the tubes were centrifuged and the clear supernatants were separated for Pi and Ca²⁺ analyses. Pi concentration was determined spectrophotometrically at 820 nm using a modification of the ammonium molybdate method of B. N. Ames, Methods of Enzymology 1966; 8:115-118. Calcium was measured calorimetrically using a modification of the O-cresolpthalein complexone microdetermination method of E. S. Baginski, et al., Clin Chim Acta 1973; 46:49-54. Calcium reacts with o-cresolphthalein complexone in the presence of 8-hydroxyquinoline to form a purple chromophore. The intensity of the final color reaction is proportional to the amount of calcium in the sample.

Thus within each sample, the seven parameters were analyzed and subsequently compared. Overall statistical analysis of differences between the various treatment parameters was performed using ANOVA with post hoc Tukey HSD multiple comparisons. Results are presented as the mean±standard error of the mean. The results of the experiments are shown in FIGS. 2A-2D.

### Example 2

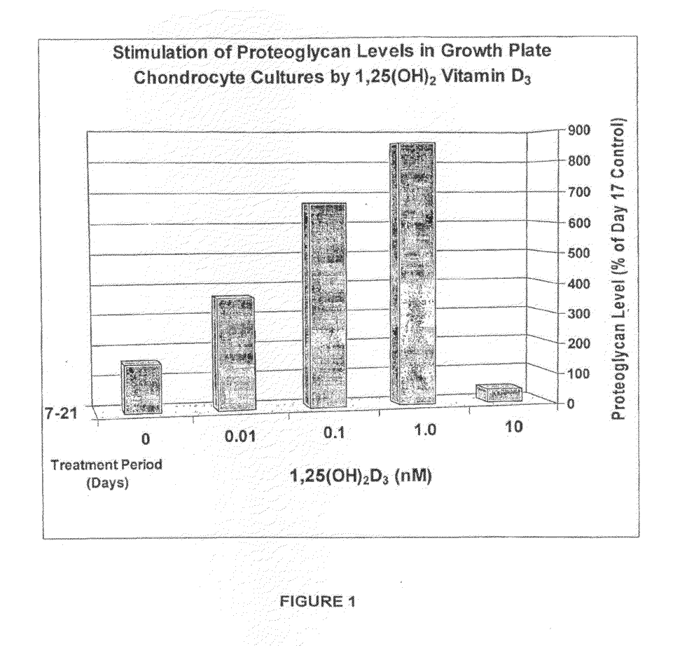

FIG. 1 demonstrates the effects of treating chondrocytes with nanomolar levels of 1,25(OH)₂D₃ and 24,25(OH)₂D₃. The protocol giving rise to the data in FIG. 1 is outlined in Example 1, above. The Vitamin D₃ treatment leads to profound increases in proteoglycan and is found to be cell-stage dependent, i.e. the effect of the Vitamin D₃ metabolites is dependent upon the stage of development of the growing cells. Therefore, this invention also has important broad applications and ramifications in guiding the growth and differentiation of stem cells and may likely be used in developing living articular cartilage equivalents and other areas of stem cell research and tissue engineering.

Proteoglycans are a specific component of all types of cartilage; in combination with type-2 collagen they are responsible for the viscoelastic properties of these tissues. They also provide an important diffusion barrier that buffers these cells from external influences.

As seen above, in control cultures grown in serum-containing DATP % medium, proteoglycan levels progressively increased with time. In serum-free HL-1 mediums, proteoglycan levels increased significantly less rapidly.

In serum-containing DATP5 medium, early exposure (Day 7-17) to 1,25(OH)₂D₃ led to a marked dosage-dependent increase in proteoglycan levels (more than four-fold over the control at 10 nM); at that time, 24,25(OH)₂D₃ caused minimal stimulation. Longer exposure to 24,25(OH)₂D₃, however, led to significant increases in proteoglycan levels, equal to or greater than those caused by 1,25(OH)₂D₃.

In serum-free HL-1 medium the effects of the Vitamin D₃ metabolites on proteoglycan levels were significantly greater. Maximal stimulation of 1,25(OH)₂D₃ was seen at −1.0 nM and ranged from −4-fold on Day 17 to nearly 7-fold by Day 24. Supra-physiological levels of 1,25(OH)₂D₃ (10 nM), however, led to marked reduction in proteoglycan levels. 24,25(OH)₂D₃ also stimulated proteoglycan formation in serum-free media. Its maximal effects, seen at 10 nM, were only about half of those with 1,25(OH)₂D₃, but still highly significant. Supra-physiological levels of 24,25(OH)₂D₃ (100 nM) were also inhibitory to proteoglycan formation in serum-free media. Thus, proteoglycan synthesis by GP chondrocytes is highly sensitive to the level of both 1,25(OH)₂D₃ and 24,25(OH)₂D₃, especially in the absence of serum which contains proteins that buffer their availability.

Post-Confluent Cultures—The most striking difference between the effect of post-confluent and pre-confluent treatment with 1,25(OH)₂D₃ was the finding that high levels were no longer inhibitory, but actually increased proteoglycan levels. This occurred in both serum-containing and serum-free cultures. In contrast, post-confluent exposure to 24,25(OH)₂D₃ caused only small effects on proteoglycan. As will be discussed later, the lack of inhibition by high levels of 1,25(OH)₂D₃ in the post-confluent state appears to result from the fact that the cells had already produced significant amounts of proteoglycan by Day 14 when the cells were first exposed to the metabolite. These studies reveal that GP cells are exquisitely sensitive to 1,25(OH)₂D₃, their responses depending on the level, timing, and medium in which exposure occurred. We would expect other cells found in joints such as articular chondrocytes, fibroblasts, synoviocytes, and cells which make up the epidermis are epithelial cells, keratinocytes, melanocytes, Langerhans cells, etc to respond similarly to Vitamin D₃ treatment by producing more proteoglycans, in both a dose and time (cell stage specific) dependent manner.

In another aspect, therefore, the invention is a method of modulating proteoglycan formulations in living animal cells, the method comprising contacting the cells with one or more Vitamin D₃ metabolites at concentrations between zero and physiological levels of the Vitamin D₃ metabolites in order to increase the production of proteoglycans in the living cells or contacting the cells with one or more Vitamin D₃ metabolites at concentrations above physiological levels in order to decrease the production of proteoglycans in the living cells.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties.

The discussion of the references herein is intended merely to summarize the assertions made by their authors and no admission is made that any reference constitutes prior art. Applicants reserve the right to challenge the accuracy and pertinency of the cited references.

Although preferred embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part.

What is claimed is:

1. A method of stimulating the production of proteoglycans in a living animal cells comprising administering one or more Vitamin D₃ metabolites to the living cells.

2. The method according to claim 1 wherein the cells are one or more of normal, rapidly dividing, healthy, healing, and diseased cells.

3. The method according to claim 1, wherein the one or more Vitamin D₃ metabolites are administered to the cells in vivo.

4. The method according to claim 1, wherein the one or more Vitamin D₃ metabolites are administered to the cells in vitro.

5. The method according to claim 1, wherein the cells are included in a subject and the one or more Vitamin D₃ metabolites are administered to the subject by enteral administration.

6. The method according to claim 5, wherein the enteral administration comprises oral administration of one or more Vitamin D₃ metabolites as a supplement to one or more of beverages and food products.

7. The method according to claim 6, wherein the beverages are one or more of milk, juice, water, and soft drinks.

8. The method according to claim 6, wherein the food products are one or more of nutritional supplements, energy bars, cereal products, genetically modified fruits, genetically modified grains, and genetically modified vegetables.

9. The method according to claim 6, wherein the one or more Vitamin D₃ metabolites are administered as one or more of a tablet and capsule.

10. The method according to claim 1, wherein the cells are included in a subject and the one or more Vitamin D₃ metabolites are administered to the subject by parenteral administration.

11. The method according to claim 1, wherein the cells are included in a subject and the one or more Vitamin D₃ metabolites are administered to the subject topically.

12. The method according to claim 1, wherein the cells are included in a subject and the administration is to the subject and is one or more of orally, subcutaneously, intranasally, intravenously, intramuscularly, buccally, and sublingually

13. The method according to claim 1, wherein the one or more Vitamin D₃ metabolites comprise 1,25(OH)₂ Vitamin D₃ metabolites.

14. The method according to claim 1, wherein the one or more Vitamin D₃ metabolites comprise 24,25(OH)₂ Vitamin D₃ metabolites.

15. The method according to claim 1, wherein between about 0.1 IU and 2400 IU of the one or more Vitamin D₃ metabolites are administered to a subject daily.

16. The method according to claim 5, wherein the subject is one that is in need of treatment for one or more of wound healing, osteoarthritis, cosmetic deficiencies, and cancer.

17. The method according to claim 1, wherein the living animal cells are one or more of chondrocytes, synoviocytes, pannocytes, and the like.

18. A method of modulating proteoglycan formation in living animal cells, the method comprising contacting the cells with one or more Vitamin D₃ metabolites at concentrations between zero and physiological levels of the Vitamin D₃ metabolites in order to increase production of proteoglycans in the cells.

19. A method of modulating proteoglycan formation in living animal cells, the method comprising contacting the cells with one or more Vitamin D₃ metabolites at concentrations above physiological levels of the Vitamin D₃ metabolites in order to decrease production of proteoglycans in the cells.

20. A method of preventing and/or treating osteoarthritis, the method comprising administering one or more Vitamin D₃ metabolites to chondrocyte cells in a subject in need of treatment for osteoarthritis and having an affected joint area to stimulate the production of proteoglycans in the chondrocyte cells.

21. A method of treating cancer, the method comprising administering a composition including Vitamin D₃ metabolites to cancer cells to stimulate the production of proteoglycans in the cancer cells.

22. A method of enhancing the rate of wound healing, the method comprising administering a composition of Vitamin D₃ metabolites to the wound.

23. The method according to claim 22, wherein the composition is administered topically to the wound location.

24. The method according to claim 23, wherein the administration step is one or more of orally, subcutaneously, intranasally, intravenously, intramuscularly, bucally, and sublingually.

25. Use of one or more Vitamin D₃ metabolites for the purpose of preparing a medicament from the prevention and treatment of osteoarthritis and cancer, and for the enhancement of wound healing, the medicament comprising one or more Vitamin D₃ metabolites and a pharmaceutically acceptable excipient.
